(19) United States
(12) Patent Application Publication
(10) Pub. No.: US 2019/0328781 A1
(43) Pub. Date: Oct. 31, 2019

(54) MANUFACTURING METHODS FOR CELL-BASED THERAPEUTIC COMPOSITIONS

(71) Applicant: Mustang Bio, Inc., New York, NY (US)

(72) Inventors: Sadik Kassim, Milton, MA (US); Kenny Choi, Los Angeles, CA (US)

(73) Assignee: Mustang Bio, Inc., New York, NY (US)

(21) Appl. No.: 16/375,696

(22) Filed: Apr. 4, 2019

Related U.S. Application Data
(60) Provisional application No. 62/654,004, filed on Apr. 6, 2018.

(51) Int. Cl. A61K 35/15; A61K 35/17; A61K 35/18; A61K 35/19

(52) U.S. Cl. CPC A61K 35/15; A61K 35/19; A61K 35/18; A61K 35/17

(57) ABSTRACT

The present disclosure relates generally to methods for preparing cell-based therapeutic compositions for treating hematological cancers. In particular, the disclosure relates to depleting CD56+ cells from cell-based therapeutics used to treat hematological cancers.

### CROSS-REFERENCE TO RELATED APPLICATION

This application claims priority from U.S. Provisional Patent Application No. 62/654,004, filed Apr. 6, 2018. The contents of this application is incorporated herein by reference in its entirety.

### FIELD OF INVENTION

The present disclosure relates generally to cell-based therapeutics and methods of making or manufacturing the same. In particular, the disclosed methods and compositions provide a novel and efficient way to treat various types of hematological cancers, including but not limited to blastic plasmacytoid dendritic cell neoplasm (BPDCN), acute myeloid leukemia (AML), or myelodysplastic syndrome (MDS). Finally, the present disclosure relates to methods of preparing cell-based therapeutics with a lower rate of failure than previously achievable using conventional preparation methods.

### BACKGROUND

For years, the foundations of cancer treatment were surgery, chemotherapy, and radiation therapy. More recently, targeted therapies like imatinib and trastuzumab—drugs that target cancer cells by homing in on specific molecular changes seen primarily in those cells—have also cemented themselves as standard treatments for many cancers. But over the past several years, immunotherapies have emerged as what many in the cancer community now call the “fifth pillar” of cancer treatment.

A rapidly emerging immunotherapy approach is relies on cell-based therapeutics, such as adoptive cell transfer (ACT). In ACT, a patients' own immune cells are collected and modified before being administered back to the patient to treat his or her cancer. There are several types of ACT, but thus far, the one that has advanced the furthest in clinical development is called chimeric antigen receptor (CAR) T-cell therapy. While two CAR T-cell therapies were approved by the Food and Drug Administration (FDA) in 2017, one for the treatment of children with acute lymphoblastic leukemia (ALL) and the other for adults with advanced lymphomas, there is still much development needed in order to ensure that CAR T-cell therapy is efficient and effective, and that the benefits of the therapy are reproducible from patient to patient.

In particular, the process for preparing cell-based therapeutics like CAR T-cells could benefit not only from standardization, but also development of methods that will increase the likelihood of success. For instance, the white blood cell fractions in the blood of individuals with certain types of hematological cancers, such as blastic plasmacytoid dendritic cell neoplasm (BPDCN), acute myeloid leukemia (AML), or myelodysplastic syndrome (MDS), are often highly contaminated by blast cells. In some cases, up to 100% of the cells in circulation can be blasts. See, e.g., Sullivan, J., Treatment of blastic plasmacytoid dendritic cell neoplasm, American Society of Hematology (2016); Laribi K., Blastic plasmacytoid dendritic cell neoplasm: From the origin of the cell to targeted therapies; Review, Biology of Blood and Marrow Transplantation (2016), doi: 10.1016/j.bbmt.2016.03.022.

This high percentage of blast cells leads to a reduced frequency of T cells, which can subsequently have an impact on the success of CAR-T manufacturing processes. Indeed, numerous clinical and experimental failures have resulted from an inability to obtain a sufficient population of healthy cells to use in the cell-based therapy.

Thus, cell-based therapeutics are of intense medical interest, particularly in relation to the treatment of hematological cancers, but a significant unmet medical need still exists for developing methods and processes for efficiently and effectively preparing the cell-based therapeutics. The present disclosure satisfies this need.

### SUMMARY

The present disclosure provides methods of preparing cell-based therapeutics that involve depleting CD56+ cells from a population of therapeutic cells, as well as cell-based therapeutic compositions that have been depleted of CD56+ cells, and methods of treating hematological cancers using the disclosed compositions and/or cell-based therapeutics prepared according to the disclosed methods of manufacturing.

In one aspect, the disclosure relates to a method of preparing a cell-based therapeutic composition useful for a treatment of a hematological cancer comprising: depleting cells that express CD56 from an apheresis sample taken from a subject diagnosed with a hematological cancer to obtain a remainder; and transducing cells in the remainder with a nucleic acid that encodes a chimeric receptor having a binding affinity for an antigen expressed by or associated with the hematological cancer.

In some embodiments, the method may further comprise fractionating cells in the remainder to obtain fractionated cells, then transducing the fractionated cells. In some embodiments, the fractionation step comprises adding a density-based separation medium (e.g., Ficoll) to the remainder to obtain a multilayered mixture after a mixing step, and collecting the fractionated cells found in an interphase between a plasma layer and a separation medium layer.

In some embodiments, the method may further comprise activating the cells in the remainder, then transducing the activated cells. In some embodiments, the activation step comprises contacting the cells with CD3, CD28, 4-1BB, CD27, ICOS, OX40, HVEM, CD30 and/or any other member of the family of T cell co-stimulatory molecules.

In some embodiments, the method may further comprise comprising culturing the transduced cells for at least 3 days, for example, at least 4, at least 5, at least 6, or at least 7 or more days.

In some embodiments, the patient may have a hematological cancer that comprises cells that over-express CD123. In some embodiments, the patient may have blastic plasmacytoid dendritic cell neoplasm (BPDCN), acute myeloid leukemia (AML), or myelodysplastic syndrome (MDS).

In some embodiments, the chimeric receptor encoded by the nucleic acid has a binding affinity for CD123, CD33, CD47, CD117, CD25, FLT-3, CXCR4, WT-1, LeY, CD56, or CD303. In some embodiments, the chimeric receptor comprises the amino acid sequence of SEQ ID NO: 1 or SEQ ID NO: 2.

In some embodiments, the chimeric receptor comprises at least one costimulatory domain (e.g., a costimulatory region of CD28, 4-1BB, CD3, CD27, ICOS, OX40, HVEM, CD30 and/or any other member of the family of T cell co-stimulatory molecules), a transmembrane domain (e.g., a transmembrane portion of CD28, CD4, CD8, 4-1BB, CD27, ICOS, OX40, HVEM, or CD30), and an antigen-binding domain, such as a scFv.

In some embodiments, the method may further comprise depleting from the apheresis sample cells that express at least one of CD4, CD123, TCL1, CD2AP, BDCA2, CD303, MPO, lysozyme, CD34, CD14, CD11c, or CD163.

In some embodiments, at least 50% of the cells in the remainder comprise peripheral blood mononuclear cells (PBMCs). In some embodiments, less than 50% of the cells in the remainder express CD56.

In another aspect, the present disclosure provides a cell-based immunotherapeutic composition comprising a population of peripheral blood mononuclear cells (PBMCs) which (i) expresses a chimeric receptor that binds to an antigen expressed by or associated with a hematological cancer, and (ii) does not substantially comprise cells that express CD56.

In some embodiments, the population of PBMCs arose from an apheresis sample taken from a subject diagnosed with the hematological cancer, and the apheresis sample was substantially depleted of cells that express CD56.

In some embodiments, the composition comprises less than about 50% blast cells. In some embodiments, the composition comprises at least 50% T-cells. In some embodiments, the PBMCs comprise T-cells. In some embodiments, the PBMCs are autologous.

In some embodiments, the hematological cancer comprises cells that over-express CD123. In some embodiments, the hematological cancer comprises blastic plasmacytoid dendritic cell neoplasm (BPDCN), acute myeloid leukemia (AML), or myelodysplastic syndrome (MDS).

In some embodiments, the chimeric receptor has a binding affinity for CD123, CD33, CD47, CD117, CD25, FLT-3, CXCR4, WT-1, LeY, CD56, or CD303, and in some embodiments the chimeric receptor comprises at least one costimulatory domain (e.g., a costimulatory region of CD28, 4-1BB, CD3, CD27, ICOS, OX40, HVEM, CD30 and/or any other member of the family of T cell co-stimulatory molecules), a transmembrane domain (e.g., a transmembrane portion of CD28, CD4, CD8, 4-1BB, CD27, ICOS, OX40, HVEM, or CD30), and an antigen-binding domain, such as a scFv.

In some embodiments, the chimeric receptor comprises the amino acid sequence of SEQ ID NO: 1 or SEQ ID NO: 2.

In some embodiments, the population of PBMCs does not substantially comprise cells that express at least one of CD4, CD123, TCL1, CD2AP, BDCA2, CD303, MPO, lysozyme, CD34, CD14, CD11c, or CD163.

In another aspect, the present disclosure provides a method of treating a patient diagnosed with blastic plasmacytoid dendritic cell neoplasm (BPDCN), acute myeloid leukemia (AML), or myelodysplastic syndrome (MDS) comprising administering to the patient in need thereof a cell-based immunotherapeutic comprising autologous PBMCs that express a chimeric receptor that binds to an antigen expressed by or associated with BPDCN, AML, or MDS and in which the cell-based immunotherapeutic does not substantially comprise cells that express CD56.

In some embodiments, the chimeric receptor binds CD123.

In some embodiments, the cell-based immunotherapeutic composition comprises less than about 50% blast cells.

In some embodiments, the cell-based immunotherapeutic does not substantially comprise cells that express at least one of CD4, CD123, TCL1, CD2AP, BDCA2, CD303, MPO, lysozyme, CD34, CD14, CD11c, or CD163.

In some embodiments, the PBMCs comprise T-cells, and in some embodiments, the PBMCs are autologous.

In some embodiments, the chimeric receptor comprises the amino acid sequence of SEQ ID NO: 1 or SEQ ID NO: 2.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIG. 1 shows a flow diagram of an exemplary process for preparing a cell-based therapeutic for treating a hematological cancer (e.g., BPDCN, AML, or MDS).

FIGS. 2A and 2B shows a conventional process for preparing CAR T-cells compared to the disclosed process. FIG. 2A shows the conventional process, while FIG. 2B shows the exemplary version of the disclosed process.

### DETAILED DESCRIPTION

The compositions and methods of the present disclosure employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds 1987, and periodic updates); PCR: The Polymerase Chain Reaction, (Mullis et al., ed., 1994); A Practical Guide to Molecular Cloning (Perbal Bernard V., 1988); Phage Display: A Laboratory Manual (Barbas et al., 2001).

### I. Definitions

It is to be understood that methods are not limited to the particular embodiments described, and as such may vary. It is also to be understood that the terminology used herein is for the purpose of describing particular embodiments only, and is not intended to be limiting. The scope of the present technology will be limited only by the appended claims.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “about” means plus or minus 10%.

As used herein, “optional” or “optionally” means that the subsequently described event or circumstance may or may not occur, and that the description includes instances where said event or circumstance occurs and instances where it does not.

As used herein, the terms “individual”, “patient”, or “subject” can be an individual organism, a vertebrate, a mammal (e.g., a bovine, a canine, a feline, or an equine), or a human. In a preferred embodiment, the individual, patient, or subject is a human.

As used herein, the terms “depletion,” “depleted,” or “depleting” mean to reduce or remove a particular cell or cell type from a larger population of cells. For instance, the disclosed methods comprise depleting an apheresis sample of cells that express CD56 (i.e., CD56+ cells). Thus, cells that express CD56 are removed from the apheresis sample, leaving behind a population of cells that largely do not express CD56. It is emphasized that depletion of a certain cell type may not remove 100% of the cell type targeted for depletion, but is expected to remove substantially all of the targeted cell type (e.g., 50, 55, 60, 65, 70, 75, 80, 85, 86, 87, 88, 89, 90, 91, 92, 93, 94, 95, 96, 97, 98, 99, or 100% of the targeted cell population).

As used herein, the phrases “therapeutically effective amount” and “therapeutic level” mean that drug dosage or plasma concentration in a subject, respectively, that provides the specific pharmacological effect for which the drug is administered in a subject in need of such treatment, i.e. to reduce, ameliorate, or eliminate the symptoms or effects of cancer, malignant disease, or cancer cell proliferation. It is emphasized that a therapeutically effective amount or therapeutic level of a drug will not always be effective in treating the conditions/diseases described herein, even though such dosage is deemed to be a therapeutically effective amount by those of skill in the art. The therapeutically effective amount may vary based on the route of administration and dosage form, the age and weight of the subject, and/or the subject's condition, including the type and stage of the cancer, malignant disease, or cancer cell proliferation, among other factors.

The terms “treatment” or “treating” as used herein with reference to cancer, malignant disease, or cancer cell proliferation refer to reducing, ameliorating or eliminating one or more symptoms or effects of cancer, malignant disease, or cancer cell proliferation.

### II. CD56 as a Marker for Depletion

CD56, also known as neural cell adhesion molecule (NCAM), is a homophilic binding glycoprotein expressed on the surface of neurons, glia, and skeletal muscle, among other cell types. Although CD56 is often considered a marker of neural lineage commitment due to its discovery site, CD56 expression is also found in, among others, the hematopoietic system, where the expression of CD56 is most stringently associated with, but certainly not limited to, natural killer cells. CD56 has been detected on other lymphoid cells, including gamma delta (γδ) T-cells and activated CD8+ T-cells, as well as on dendritic cells.

For the purposes of this disclosure, it has been discovered that CD56 expression is associated with numerous hematological cancers, including but not limited to, blastic plasmacytoid dendritic cell neoplasm (BPDCN), acute myeloid leukemia (AML), myelodysplastic syndrome (MDS), myeloma, myeloid leukemia, and NK/T cell lymphomas, among others.

As disclosed in more detail below, the present application provides methods of producing cell-based immunotherapies in which an autologous apheresis sample is taken from a patient with a hematological cancer (e.g., BPDCN, AML, MDS, etc.) and cells expressing CD56 are depleted from the apheresis sample prior to introducing a nucleotide sequence that encodes a chimeric receptor into the remaining cells. The present inventors have found that this improves production efficiency and ultimately leads to a more effective immunotherapeutic composition.

### III. Methods for Producing Cell-Based Therapeutic Compositions

Provided herein are processing strategies for reducing the disease burden (i.e., blast cell population) in the starting patient material (i.e., an apheresis sample) used to produce a cell-based immunotherapy. The disclosed methods increase process control and overall manufacturability. CD56 is a relevant surface antigen for identifying BPDCN, AML, and MDC blasts, among other types of hematological cancers, and it can serve as a target for depleting the blast cells from the starting apheresis sample. Indeed, undesirable blast cells will be the primary cell type to express CD56 in an apheresis sample, and therefore depleting CD56-expressing cells (i.e., blast cells) from the apheresis sample prior to transduction with a nucleic acid encoding a chimeric receptor, the disclosed process provides a way to dramatically improve the cell-based immunotherapeutic production process.

Accordingly, in one aspect the present disclosure provides methods of preparing a cell-based therapeutic composition useful for a treatment of a hematological cancer comprising: depleting cells that express CD56 from an apheresis sample taken from a subject diagnosed with a hematological cancer to obtain a remainder; and transducing cells in the remainder with a nucleic acid that encodes a chimeric receptor having a binding affinity for an antigen expressed by or associated with the hematological cancer.

The disclosed process may further comprise fractionating cells in the remainder to obtain fractionated cells, then transducing the fractionated cells. Those of ordinary skill in the art will understand that fractionation can be performed using a variety of conventional methods. For instance, a fractionation step may comprise adding a density-based separation medium to the remainder to obtain a multilayered mixture. After a mixing the density-based separation medium with the remainder, multiple fractionated layers may form, and the fractionated cells found in an interphase between a plasma layer and a separation medium layer can be collected, thus further enriching the remainder with a population of cells that is well-suited for transduction with a chimeric receptor. Various density-based separation mediums are known in the art, such as Ficoll®. Thus, subsequent to the depletion of CD56-expressing blasts from the apheresis sample, the remainder can be “ficolled” to reduce red blood cells (RBCs) and polymorphic cells. It may be advantageous to perform the ficollation post-depletion of the blast cells as an augmented apheresis will likely further improve the performance of the PBMC enrichment.

The disclosed process may also comprise activating the cells in the remainder prior to transduction, thereby only transducing activated cells. Those of ordinary skill in the art will understand that activation of the cells in the remainder (e.g., T-cells) can be performed using a variety of conventional methods. For instance, some common methods of activation include contacting T-cells with CD28 and/or CD3, however, activation may be achieved by contacting the cells with other co-stimulatory factors, including but not limited to, 4-1BB, CD27, ICOS, OX40, HVEM, CD30 and/or any other member of the family of T cell co-stimulatory molecules. Accordingly, these co-stimulatory factors may be used alone or in combination to activate the cells in the remainder of the disclosed process.

Generally, once the cells in the disclosed process have been transduced with a nucleic acid that encodes a chimeric receptor, the cells will be cultured for a period of time in order to expand the cell population to a useful size (i.e., to provide a sufficient number of cells to treat a hematological cancer). Thus, the disclosed process may further comprise a step of culturing the transduced cells for about 1-20 days, about 2-18 days, about 3-15 days, or about 7-10 days. For examples, the cells may be cultured for at least about 1, 2, 3, 4, 5, 6, 7, 8, 9, 10, 11, 12, 13, 14, 15, 16, 17, 18, 19, or 20 days.

The disclosed process can be used for preparing a variety of cell-based immunotherapies that can treat a variety of hematological cancers. For example, in some embodiments, the patient from which the apheresis sample was obtained may have a hematological cancer that comprises cells that over-express CD123, CD33, CD47, CD117, CD25, FLT-3, CXCR4, WT-1, LeY, CD56, or CD303. In some embodiments, the patient from which the apheresis sample was obtained may have blastic plasmacytoid dendritic cell neoplasm (BPDCN), acute myeloid leukemia (AML), or myelodysplastic syndrome (MDS).

The chimeric receptor that is encoded by the nucleic acid transduced into the CD56-depleted cell population is not particularly limited, and the chimeric receptor may be designed to bind to any relevant pathological marker of a hematological marker. For instance, in some embodiments the chimeric receptor may have a binding affinity for CD123, CD33, CD47, CD117, CD25, FLT-3, CXCR4, WT-1, LeY, CD56, or CD303.

The overall structure of the chimeric receptor that is expressed CD56-depleted cell population is likewise not particularly limited, but will generally comprise at least a costimulatory domain (or domains), a transmembrane domain, and an antigen-binding domain. Exemplary costimulatory domains may comprise, but are not necessarily limited to, a costimulatory region of CD28, 4-1BB, CD3, CD27, ICOS, OX40, HVEM, CD30 and/or any other member of the family of T cell co-stimulatory molecules. Exemplary transmembrane domains comprise, but are not necessarily limited to, a transmembrane portion of CD28, CD4, CD8, 4-1BB, CD27, ICOS, OX40, HVEM, or CD30. Exemplary antigen-binding domains may comprise, but are not necessarily limited to an scFv.

In some embodiments, the chimeric receptor comprises the amino acid sequence of SEQ ID NO: 1 or SEQ ID NO: 2, as shown in the Table below. In some embodiments, the chimeric receptor comprises the variable heavy (VH) binding domain sequence and/or a variable light (VL) binding domain sequence of SEQ ID NOs: 1 or 2. In some embodiments, the chimeric receptor comprises the complementarity determining regions (CDRs) of the scFv disclosed in SEQ ID NOs: 1 or 2.

TABLE1

| | SEQ | |
|---|---|---|
| | ID NO: | Sequence |
| Anti- | 1 | MLLLVTSLLLCELPHPAFLLIPQVQLQQPGAELVRPGASVKLSCK |
| CD123 | | ASGYTFTSYWMNWVKQRPDQGLEWIGRIDPYDSETHYNQKFKD |
| CAR1 | | KAILTVDKSSSTAYMQLSSLTSEDSAVYYCARGNWDDYWGQGT |
| | | TLTVSSGGGGSGGGGSGGGGSDVQITQSPSYLAASPGETITINCRA |
| | | SKSISKDLAWYQEKPGKTNKLLIYSGSTLQSGIPSRFSGSGSGTDF |
| | | TLTISSLEPEDFAMYYCQQHNKYPYTFGGGTKLEIKESKYGPPCPP |
| | | CPAPEFEGGPSVFLFPPKPKDTLMISRTPEVTCVVVDVSQEDPEVQ |
| | | FNWYVDGVEVHNAKTKPREEQFQSTYRVVSVLTVLHQDWLNG |
| | | KEYKCKVSNKGLPSSIEKTISKAKGQPREPQVYTLPPSQEEMTKN |
| | | QVSLTCLVKGFYPSDIAVEWESNGQPENNYKTTPPVLDSDGSFFL |
| | | YSRLTVDKSRWQEGNVFSCSVMHEALHNHYTQKSLSLSLGKMF |
| | | WVLVVVGGVLACYSLLVTVAFIIFWVRSKRSRGGHSDYMNMTP |
| | | RRPGPTRKHYQPYAPPRDFAAYRSGGGRVKFSRSADAPAYQQGQ |
| | | NQLYNELNLGRREEYDVLDKRRGRDPEMGGKPRRKNPQEGLYN |
| | | ELQKDKMAEAYSEIGMKGERRRGKGHDGLYQGLSTATKDTYDA |
| | | LHMQALPPR |
| Anti- | 2 | MLLLVTSLLLCELPHPAFLLIPQIQLVQSGPELKKPGETVKISCKAS |
| CD123 | | GYIFTNYGMNWVKQAPGKSFKWMGWINTYTGESTYSADFKGRF |
| CAR2 | | AFSLETSASTAYLHINDLKNEDTATYFCARSGGYDPMDYWGQGT |
| | | SVTVSSGGGGSGGGGSGGGGSDIVLTQSPASLAVSLGQRATISCR |
| | | ASESVDNYGNTFMHWYQQKPGQPPKLLIYRASNLESGIPARFSGS |
| | | GSRTDFTLTINPVEADDVATYYCQQSNEDPPTFGAGTKLELKESK |
| | | YGPPCPPCPAPEFEGGPSVFLFPPKPKDTLMISRTPEVTCVVVDVS |
| | | QEDPEVQFNWYVDGVEVHNAKTKPREEQFQSTYRVVSVLTVLH |
| | | QDWLNGKEYKCKVSNKGLPSSIEKTISKAKGQPREPQVYTLPPSQ |
| | | EEMTKNQVSLTCLVKGFYPSDIAVEWESNGQPENNYKTTPPVLD |
| | | SDGSFFLYSRLTVDKSRWQEGNVFSCSVMHEALHNHYTQKSLSL |
| | | SLGKMFWVLVVVGGVLACYSLLVTVAFIIFWVRSKRSRGGHSDY |
| | | MNMTPRRPGPTRKHYQPYAPPRDFAAYRSGGGRVKFSRSADAPA |
| | | YQQGQNQLYNELNLGRREEYDVLDKRRGRDPEMGGKPRRKNPQ |
| | | EGLYNELQKDKMAEAYSEIGMKGERRRGKGHDGLYQGLSTATK |
| | | DTYDALHMQALPPR |

In addition to depletion of CD56+ cells from the apheresis sample, additional benefit may be obtained by depleting cells that express other markers, for example, markers that are commonly expressed on blast or cancerous cells. Accordingly, in some embodiments, the disclosed process may further comprise depleting from the apheresis sample cells that express at least one of CD4, CD123, TCL1, CD2AP, BDCA2, CD303, MPO, lysozyme, CD34, CD14, CD11c, or CD163. In particular, depleting cells that express at least one of CD123, TCL1, CD2AP, or BDCA2 may improve the therapeutic viability of the population of cells that are to be transduced. These additional depletion targets are shown in the Table below, which shows the percent of cells expressing certain shared markers and the markers unique to the given cell type.

TABLE 2

| | | BPDCN | AML/LC/MS |
|---|---|---|---|
| SHARED | CD4 | 80-100% | 10-20% |
| | CD56 | 90-100% | 5-50% |
| | CD123 | 85-100% | 15-45% |
| | TCL1 | 80-100% | 5-20% |

| | | |
|---|---|---|
| UNIQUE | CD2AP | MPO |
| | CD303/BDCA-2 | Lysozyme |
| | | CD34 |
| | | CD14 |
| | | CD11c |
| | | CD163 |

In some embodiments, the markers shown in the foregoing Table may be used as targets for depleting further undesirable cells form the starting apheresis sample in order to further improve the disclosed methods of production.

In some embodiments of the disclosed process, after the apheresis sample has been depleted of CD56+ cells, the majority of the cells in the remainder may comprise peripheral blood mononuclear cells (PBMCs). For example, the remainder may comprise at least about 50%, at least about 55%, at least about 60%, at least about 65%, at least about 70%, at least about 75%, at least about 80%, at least about 85%, at least about 90%, at least about 95% or more PBMCs.

In some embodiments, depletion of cells expressing CD56 (i.e., CD56+ cells) will result in a population of cells in the remained that has fewer cells expressing CD56 than the starting apheresis sample. For example, the depletion step may reduce the number of cells expressing CD56 by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more compared to the starting apheresis sample. In some embodiments, the disclosed process will provide a remainder that may comprise less than about 50, less than about 45, less than about 40, less than about 35, less than about 30, less than about 25, less than about 20, less than about 15, less than about 10, or less than about 5% cells expressing CD56. Additionally or alternatively, in some embodiments, the disclosed process will provide a remainder that may comprise less than about 50, less than about 45, less than about 40, less than about 35, less than about 30, less than about 25, less than about 20, less than about 15, less than about 10, or less than about 5% blast cells.

Those of skill in the art known that there are various cell processing, separating, and sorting instruments that could be used to deplete cells expressing CD56 from an apheresis sample, such as, for example, a CLINIMACS® system. Existing cell depletion methods that are active in GMP manufacturing can enable CD56 depletion without changes to the method, and a clinical grade CD56 reagent is already available off-the-shelf (see, e.g., miltenyibiotec.com/en/clinical-applications/clinimacs-system/clinimacs-reagents/clinimacs-cd56-product-line.aspx). Furthermore, the starting T-cell phenotype will not be impacted by this process improvement as only cell populations that are non-relevant to the final product will be manipulated. The starting T-cell population will therefore be equivalent to the current state and will not require further characterization.

Without being bound by theory, it is believed that as a result of the improved efficiency of the disclosed production process, fewer cells may be administered to a patient receiving ACT. This is because a cell-based immunotherapeutic produced by the disclosed methods will contain a higher relative amount of T-cells and fewer undesirable blast cells. As a result, a cell-based immunotherapy produced according to the proposed methods may be considered more potent than a cell-based immunotherapy that did not undergo CD56 depletion.

### IV. Cell-Based Immunotherapeutic Compositions

Provided herein are cell-based immunotherapeutic compositions comprising a population of peripheral blood mononuclear cells (PBMCs) which (i) express a chimeric receptor that binds to an antigen expressed by or associated with a hematological cancer, and (ii) does not substantially comprise cells that express CD56. The phrase “does not substantially comprise” may be understood as meaning the compositions contain less than about 50, less than about 45, less than about 40, less than about 35, less than about 30, less than about 25, less than about 20, less than about 15, less than about 10, or less than about 5% cells expressing CD56. Additionally or alternatively, in some embodiments, the disclosed process will provide a remainder that may comprise less than about 50, less than about 45, less than about 40, less than about 35, less than about 30, less than about 25, less than about 20, less than about 15, less than about 10, or less than about 5% of cells that express CD56

In some embodiments, the population of PBMCs arose from an apheresis sample taken from a subject diagnosed with the hematological cancer, and in some embodiments, the apheresis sample was substantially depleted of cells that express CD56. The hematological cancer may be blastic plasmacytoid dendritic cell neoplasm (BPDCN), acute myeloid leukemia (AML), or myelodysplastic syndrome (MDS) or any hematological cancer that comprises cells that over-express CD123.

In some embodiments, the PBMCs comprise T-cells. Indeed, T-cells may account for at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more of the cells in the PBMCs. In some embodiments, the PBMCs are autologous.

In some embodiments, the chimeric receptor expressed by the cells in the cell-based immunotherapy may have a binding affinity for CD123, CD33, CD47, CD117, CD25, FLT-3, CXCR4, WT-1, LeY, CD56, or CD303. In particular, the chimeric receptor may comprise the amino acid sequence of SEQ ID NO: 1 or SEQ ID NO: 2. In some embodiments, the chimeric receptor comprises the variable heavy (VH) binding domain sequence and/or a variable light (VL) binding domain sequence of SEQ ID NOs: 1 or 2. In some embodiments, the chimeric receptor comprises the complementarity determining regions (CDRs) of the scFv disclosed in SEQ ID NOs: 1 or 2.

The overall structure of the chimeric receptor that is expressed CD56-depleted cell-based immunotherapeutic not particularly limited, but will generally comprise at least a costimulatory domain (or domains), a transmembrane domain, and an antigen-binding domain. Exemplary costimulatory domains may comprise, but are not necessarily limited to, a costimulatory region of CD28, 4-1BB, CD3, CD27, ICOS, OX40, HVEM, CD30 and/or any other member of the family of T cell co-stimulatory molecules. Exemplary transmembrane domains comprise, but are not necessarily limited to, a transmembrane portion of CD28, CD4, CD8, 4-1BB, CD27, ICOS, OX40, HVEM, or CD30. Exemplary antigen-binding domains may comprise, but are not necessarily limited to an scFv.

In addition to not substantially comprising cells that express CD56 the cell-based immunotherapeutic may also be depleted of cells that express other markers, for example, markers that are commonly expressed on blast or cancerous cells. Accordingly, in some embodiments, the cell-based immunotherapeutic may not comprise cells that express at least one of CD4, CD123, TCL1, CD2AP, BDCA2, CD303, MPO, lysozyme, CD34, CD14, CD11c, or CD163. In particular, depleting cells that express at least one of CD123, TCL1, CD2AP, or BDCA2 may improve the therapeutic viability of the cell-based immunotherapeutic.

### V. Pharmaceutical Compositions and Formulations

Pharmaceutical compositions suitable for use in the methods of treatment described herein can include a cell-based therapeutic (e.g., an anti-CD123 CAR T-cell therapy depleted of CD56+ cells) and a pharmaceutically acceptable carrier or diluent.

The composition may be formulated for intravenous, subcutaneous, intraperitoneal, intramuscular, oral, nasal, pulmonary, ocular, vaginal, or rectal administration. In some embodiments, the disclosed cell-based therapeutics are formulated for intravenous, subcutaneous, intraperitoneal, or intramuscular administration, such as in a solution, suspension, emulsion, etc.

Pharmacologically acceptable carriers for various dosage forms are known in the art. For example, excipients, lubricants, solvents, solubilizing agents, suspending agents, isotonicity agents, buffers, and soothing agents are known. In some embodiments, the pharmaceutical compositions include one or more additional components, such as one or more preservatives, antioxidants, stabilizing agents and the like.

Additionally, the disclosed pharmaceutical compositions can be formulated as a solution, or other ordered structure suitable for an injection or intravenous administration. The carrier can be a solvent or dispersion medium containing, for example, water, saline solution, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In some embodiments, it will be preferable to include isotonic agents, for example, sugars, polyalcohols such as mannitol, sorbitol, or sodium chloride in the composition.

Sterile injectable solutions can be prepared by incorporating the active compound in the required amount in an appropriate solvent with one or a combination of ingredients enumerated above, and may be optionally followed by sterilization microfiltration. Generally, dispersions are prepared by incorporating the cell-based therapeutic into a sterile vehicle that contains, for example, a neutral or basic dispersion medium and the required other ingredients from those enumerated above.

Pharmaceutical compositions of the disclosure can be administered in combination with other therapeutics. For example, the combination therapy can include a pharmaceutical composition comprising at least one cell-based therapeutic with at least one or more additional therapeutic agents, including but not limited to, other CAR T-cells (e.g., modified T cells that express an anti-CD19, anti-Her2, anti-BCMA, anti-CS-1, anti-PSCA, anti-CAIX, anti-IL13R, or anti-PD-L1 CAR), tumor-targeting antibodies (e.g., an anti-CAIX, anti-PD-L1, anti-CD19, or anti-CD20 antibody), immune response potentiating modalities (e.g., an anti-GITR antibody, an anti-OX40 antibody, an anti-CD137 antibody, or a TLR agonist), and small molecule drugs (e.g., a BTK inhibitor, an EGFR inhibitor, a BET inhibitor, a PI3Kdelta inhibitor, a BRAF inhibitor, or a PARP inhibitor). Additional non-limiting examples of small molecule drugs that may be administered with the disclosed cell-based therapeutics include cladribine (LEUSTATIN®, 2-CdA), fludarabine (FLUDARA®), topotecan, etoposide (VP-16), 6-thioguanine (6-TG), hydroxyurea (HYDREA®), corticosteroid drugs (such as prednisone or dexamethasone (DECADRON®)), methotrexate (MTX), 6-mercaptopurine (6-MP), azacitidine (VIDAZA®), and decitabine. The pharmaceutical compositions of the disclosure can also be administered in conjunction with radiation therapy.

### VI. Methods of Treating Hematological Cancer

Provided herein are methods of treating hematological cancer, malignant disease, or cancer cell proliferation with the disclosed cell-based therapeutic (e.g., an anti-CD123 CAR T-cell therapy depleted of CD56+ cells). More specifically, the disclosure provides for methods of enhancing cell-based therapy function and anti-cancer efficacy comprising administering a therapeutically effective amount of any of the above described cell-based therapeutics in which CD56+ cells have been depleted from the population of cells comprised in the therapeutic.

Enhancing cell-based therapy function and anti-cancer efficacy such therapeutics of provides a benefit for treating numerous types of cancer, but is believed to be particularly beneficial for treating hematological cancers including but not limited to blastic plasmacytoid dendritic cell neoplasm (BPDCN), acute myeloid leukemia (AML), myelodysplastic syndrome (MDS), lymphoma, Non-Hodgkin's lymphoma, chronic lymphocytic leukemia (CLL), chronic myeloid leukemia (CML), acute lymphoblastic leukemia (ALL), and multiple myeloma. In some embodiments, the hematological cancer is blastic plasmacytoid dendritic cell neoplasm (BPDCN), acute myeloid leukemia (AML), or myelodysplastic syndrome (MDS). And in some embodiments, the cancer being treated according to the disclosed methods is a cancer that expresses CD123.

Dosage regimens are adjusted to provide the optimum desired response (e.g., a therapeutic response like disease regression or remission). For example, in some embodiments, a single bolus of the disclosed cell-based therapeutics may be administered, while in some embodiments, several divided doses may be administered over time or the dose may be proportionally reduced or increased as indicated by the situation. For example, in some embodiments the disclosed cell-based therapeutics may be administered once or twice weekly by, for example, intravenous injection. In some embodiments, the disclosed cell-based therapeutics may be administered once or twice monthly by subcutaneous injection. In some embodiments, the disclosed cell-based therapeutics may be administered once every week, once every other week, once every three weeks, once every four weeks, once every other month, once every three months, once every four months, once every five months, or once every six months.

Exemplary doses can vary according to the size and health of the individual being treated, as well as the condition being treated and the severity of the condition. Those of skill in the art will understand that dosing of cell-based immunotherapies may be based on (i) the fraction of CAR-positive cells/weight of the patient, (ii) an absolute number of CAR-positive cells, or (iii) an absolute number of T-cells. For example, in some embodiments, the disclosed cell-based therapeutics may be administered in a dose of 25-750 million CAR-positive T-cells. In some embodiments, the disclosed cell-based therapeutics may be administered in a dose of about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, about 500, about 505, about 510, about 515, about 520, about 525, about 530, about 535, about 540, about 545, about 550, about 555, about 560, about 565, about 570, about 575, about 580, about 585, about 590, about 595, about 600, about 605, about 610, about 615, about 620, about 625, about 630, about 635, about 640, about 645, about 650, about 655, about 660, about 665, about 670, about 675, about 680, about 685, about 690, about 695, about 700, about 705, about 710, about 715, about 720, about 725, about 730, about 735, about 740, about 745, or about 750 million CAR-position T-cells. Similar doses based on the fraction of CAR-positive cells/weight of the patient and absolute number of T-cells can also be calculated.

Furthermore, the disclosed methods of treatment can additionally comprise the administration of a second therapeutic compound in addition to the disclosed cell-based therapeutics. For example, in some embodiments, the additional therapeutic compound may be a CAR-T cell, a tumor-targeting antibody, an immune response potentiating modality, or a small molecule drug, as discussed in more detail above in the Pharmaceutical Compositions section.

Particular treatment regimens may be evaluated according to whether it will improve a given patient's outcome, meaning it will reduce the risk of recurrence or increase the likelihood of progression-free survival of the given cancer (e.g., BPDCN, AML, or MDS).

Thus, for the purposes of this disclosure, a subject is treated if one or more beneficial or desired results, including desirable clinical results, are obtained. For example, beneficial or desired clinical results include, but are not limited to, one or more of the following: decreasing one or more symptoms resulting from the cancer, increasing the quality of life of those suffering from the cancer, decreasing the dose of other medications required to treat the cancer, delaying the progression of the cancer, and/or prolonging survival of individuals.

Furthermore, while the subject of the methods is generally a haematological cancer patient, the age of the patient is not limited. The disclosed methods are useful for treating haematological cancer, malignant disease, or cancer cell proliferation with various recurrence and prognostic outcomes across all age groups and cohorts. Thus, in some embodiments, the subject may be a pediatric subject, while in other embodiments, the subject may be an adult subject.

The following examples are given to illustrate the present invention. It should be understood, however, that the invention is not to be limited to the specific conditions or details described in these examples.

### EXAMPLES

### Example 1—Production of CD56-Depleted CAR T-Cells

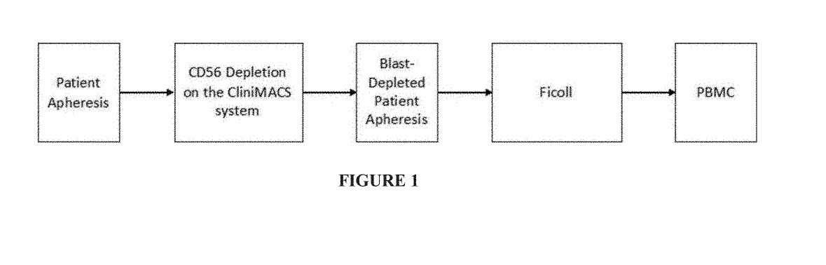

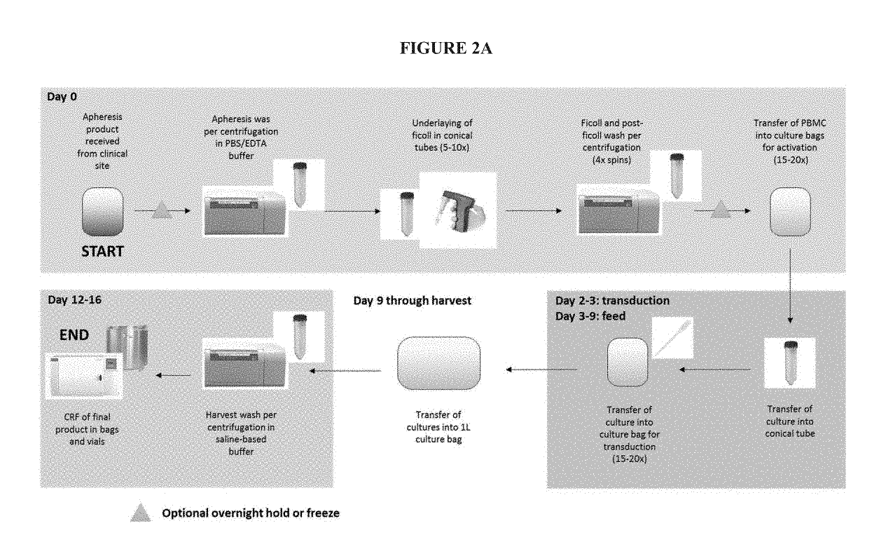

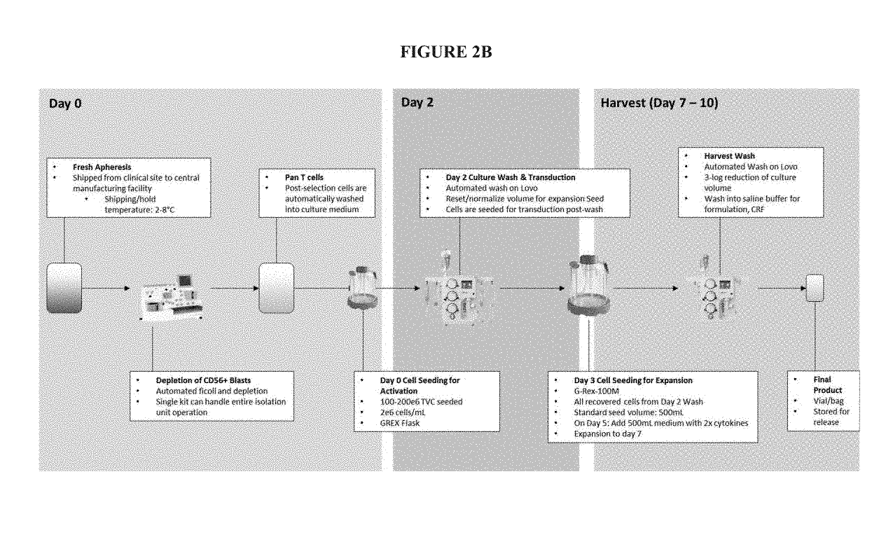

This example illustrates methods of producing cell-based therapeutics with reduced blast content and improved activity. A flow chart of an exemplary process is provided in FIG. 1.

A patient with BPDCN, AML, MDS, or another hematological cancer provides a blood sample. The blood sample from the patient undergoes apheresis to isolate immune cells. The patient apheresis sample is then depleted of cells expressing CD56 (i.e., CD56+ cells) using, for example, a CLINIMACS® system. The cells expressing CD56 in the sample should primarily be undesirable blast cells.

The blast-depleted apheresis sample is optionally “ficolled” to reduce red blood cells and polymorphic cells. These steps will provide a relatively pure population of PBMCs that can be transduced with a nucleic acid encoding a chimeric receptor.

All patents and publications mentioned in the specification are indicative of the levels of those of ordinary skill in the art to which the disclosure pertains. All patents and publications are herein incorporated by reference to the same extent as if each individual publication was specifically and individually indicated to be incorporated by reference.

Further, one skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting embodiments of the disclosure.

What is claimed:

1. A method of preparing a cell-based therapeutic composition useful for a treatment of a hematological cancer comprising:
a. depleting cells that express CD56 from an apheresis sample taken from a subject diagnosed with a hematological cancer to obtain a remainder;
b. transducing cells in the remainder with a nucleic acid that encodes a chimeric receptor having a binding affinity for an antigen expressed by or associated with the hematological cancer.

2. The method of claim 1 further comprising fractionating cells in the remainder to obtain fractionated cells, then transducing the fractionated cells.

3. The method of claim 2 in which the fractionation step comprises adding a density-based separation medium to the remainder to obtain a multilayered mixture after a mixing step, and collecting the fractionated cells found in an interphase between a plasma layer and a separation medium layer.

4. The method of claim 1 further comprising activating the cells in the remainder, then transducing the activated cells.

5. The method of claim 4 in which the activation step comprises contacting the cells with CD3, CD28, 4-1BB, CD27, ICOS, OX40, HVEM, CD30 and/or any other member of the family of T cell co-stimulatory molecules.

6. The method of claim 1 further comprising culturing the transduced cells for at least 3 days.

7. The method of claim 1 in which the hematological cancer comprises cells that over-express CD123.

8. The method of claim 1 in which the hematological cancer comprises blastic plasmacytoid dendritic cell neoplasm (BPDCN), acute myeloid leukemia (AML), or myelodysplastic syndrome (MDS).

9. The method of claim 1 in which the chimeric receptor has a binding affinity for CD123, CD33, CD47, CD117, CD25, FLT-3, CXCR4, WT-1, LeY, CD56, or CD303.

10. The method of claim 1 in which the chimeric receptor comprises at least one costimulatory domain, a transmembrane domain, and an antigen-binding domain.

11. The method of claim 10 in which the at least one costimulatory domain comprises a costimulatory region of CD28, 4-1BB, CD3, CD27, ICOS, OX40, HVEM, CD30 and/or any other member of the family of T cell co-stimulatory molecules.

12. The method of claim 10 in which the transmembrane domain comprises a transmembrane portion of CD28, CD4, CD8, 4-1BB, CD27, ICOS, OX40, HVEM, or CD30.

13. The method of claim 10 in which the antigen-binding domain comprising an scFv.

14. The method of claim 1 in which the chimeric receptor comprises the amino acid sequence of SEQ ID NO: 1 or SEQ ID NO: 2.

15. The method of claim 1 further comprising depleting from the apheresis sample cells that express at least one of CD4, CD123, TCL1, CD2AP, BDCA2, CD303, MPO, lysozyme, CD34, CD14, CD11c, or CD163.

16. The method of claim 1 further comprising depleting from the apheresis sample cells that express at least one of CD123, TCL1, CD2AP, or BDCA2.

17. The method of claim 1 in which at least 50% of the cells in the remainder comprise peripheral blood mononuclear cells (PBMCs).

18. A cell-based immunotherapeutic composition comprising a population of peripheral blood mononuclear cells (PBMCs) which (i) expresses a chimeric receptor that binds to an antigen expressed by or associated with a hematological cancer, and (ii) does not substantially comprise cells that express CD56.

19. The cell-based immunotherapeutic composition of claim 18 in which the population of PBMCs arose from an apheresis sample taken from a subject diagnosed with the hematological cancer, and the apheresis sample was substantially depleted of cells that express CD56.

20. The cell-based immunotherapeutic composition of claim 18 which comprises less than about 50% blast cells.

21. The cell-based immunotherapeutic composition of claim 18 in which the PBMCs comprise T cells.

22. The cell-based immunotherapeutic composition of claim 18 in which the hematological cancer comprises cells that over-express CD123.

23. The cell-based immunotherapeutic composition of claim 18 in which the hematological cancer comprises blastic plasmacytoid dendritic cell neoplasm (BPDCN), acute myeloid leukemia (AML), or myelodysplastic syndrome (MDS).

24. The cell-based immunotherapeutic composition of claim 18 in which the chimeric receptor has a binding affinity for CD123, CD33, CD47, CD117, CD25, FLT-3, CXCR4, WT-1, LeY, CD56, or CD303.

25. The cell-based immunotherapeutic composition of claim 18 in which the chimeric receptor comprises at least one costimulatory domain, a transmembrane domain, and an antigen-binding domain.

26. The cell-based immunotherapeutic composition of claim 25 in which the at least one costimulatory domain comprises a costimulatory region of CD28, 4-1BB, CD3, CD27, ICOS, OX40, HVEM, CD30 and/or any other member of the family of T cell co-stimulatory molecules.

27. The cell-based immunotherapeutic composition of claim 25 in which the transmembrane domain comprises the transmembrane portion of CD28, CD4, CD8, 4-1BB, CD27, ICOS, OX40, HVEM, or CD30.

28. The cell-based immunotherapeutic composition of claim 25 in which the antigen-binding domain comprising an scFv.

29. The cell-based immunotherapeutic composition of claim 18 in which the chimeric receptor comprises the amino acid sequence of SEQ ID NO: 1 or SEQ ID NO: 2.

30. The cell-based immunotherapeutic composition of claim 18 in which the population of PBMCs does not substantially comprise cells that express at least one of CD4, CD123, TCL1, CD2AP, BDCA2, CD303, MPO, lysozyme, CD34, CD14, CD11c, or CD163.

31. The cell-based immunotherapeutic composition of claim 18 in which the population of PBMCs does not substantially comprise cells that express at least one of CD123, TCL1, CD2AP, or BDCA2.

32. The cell-based immunotherapeutic composition of claim 18 in which the population of PBMCs comprises autologous cells.

33. A method of treating a patient diagnosed with blastic plasmacytoid dendritic cell neoplasm (BPDCN), acute myeloid leukemia (AML), or myelodysplastic syndrome (MDS) comprising administering to the patient in need thereof a cell-based immunotherapeutic comprising autologous PBMCs that express a chimeric receptor that binds to an antigen expressed by or associated with BPDCN, AML, or MDS and in which the cell-based immunotherapeutic does not substantially comprise cells that express CD56.

34. The method of claim 33 in which the chimeric receptor binds CD123.

35. The method of claim 32 in which the cell-based immunotherapeutic composition comprises less than about 50% blast cells.

36. The method of claim 33 in which the cell-based immunotherapeutic does not substantially comprise cells that express at least one of CD4, CD123, TCL1, CD2AP, BDCA2, CD303, MPO, lysozyme, CD34, CD14, CD11c, or CD163.

37. The method of claim 33 in which the PBMCs comprise T cells.

38. The method of claim 33 in which the chimeric receptor comprises the amino acid sequence of SEQ ID NO: 1 or SEQ ID NO: 2.


SEQUENCE LISTING (2 sequences)

SEQ ID NO: 1 - protein, 672 aa, Artificial Sequence
  Description of Artificial Sequence Synthetic polypeptide
MLLLVTSLLLCELPHPAFLLIPQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 679 aa, Artificial Sequence
  Description of Artificial Sequence Synthetic polypeptide
MLLLVTSLLLCELPHPAFLLIPQIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR